(19) United States
(12) Patent Application Publication
(10) Pub. No.: US 2003/0153824 A1
(43) Pub. Date: Aug. 14, 2003

(54) Biological information sensing device

(76) Inventor: Keisuke Tsubata, Chiba-shi (JP)

Correspondence Address: ADAMS & WILKS, ATTORNEYS AND COUNSELORS AT LAW, 31st FLOOR, 50 BROADWAY, NEW YORK, NY, 10004, US

(21) Appl. No.: 10/349,886

(22) Filed: Jan. 23, 2003

(30) Foreign Application Priority Data
Jan. 28, 2002 (JP) 2002-018258

(51) Int. Cl. A61B 5/05

(52) U.S. Cl. 600/407

(57) ABSTRACT

A biological information sensing device capable of minimizing the variations of the support for a sensor portion or the displacement thereof despite the exposure of the device to accelerative motion during exercise or regardless of the size of a neck-like portion of a user is provided. A biological information sensing device applied to a neck-like portion, including a predetermined measurement area, of a living organism as holding a sensor body thereof in intimate contact with the predetermined area for sensing biological information of a living organism, the device comprises a flexible sensor structure including the sensor body and a flexible strap-like sensor holder for holding the sensor body; and a strap for fastening the sensor structure to the neck-like portion of the living organism, and is characterized in that an engaging portion of the strap or an engaged portion of the sensor holder extends along a longitudinal direction of the strap for permitting the engaged portion of the sensor holder of the sensor structure to be engagedly secured to the engaging portion of the strap at an optional position along the longitudinal direction of the strap.

### BACKGROUND OF THE INVENTION

1. Field of the Invention

The present invention relates to a biological information sensing device for sensing biological information such as arterial pulses or the like, and more particularly, to a biological information sensing device of a type like an arterial pulse wave detector which is wrapped around the wrist or the like.

2. Description of the Related Art

A portable arterial pulse wave detector has been proposed which is provided with a pressure forming elastic piece at an intermediate portion of a strap such that the elastic piece may press a sensor portion against an area of the wrist surface near the radial artery of the wrist for holding the sensor portion in intimate contact with the wrist surface area (JP-A-8-52118).

However, the proposed portable arterial pulse wave detector has a drawback that precise measurements of arterial pulses cannot be obtained. This is because when its main body (head portion) unifying a display panel and processing circuit of great masses is subjected to a relatively great accelerative motion associated with the motion of the wrist during exercise or training, for example, an inadvertent inertia force apt to rotate/displace the head portion about the wrist is applied to the head portion, thus varying support for the sensor portion or the pressure on the sensor portion connected with the head portion for pressing the sensor portion against the wrist surface and causing the rotation/displacement of the sensor.

On the other hand, another portable arterial pulse wave detector has been proposed which has an arrangement wherein a sensor portion is adapted for slidable movement relative to a strap integral with a display portion and the like of the detector along the extension direction of the strap thereby permitting the position of the sensor portion to be adjusted according to a wrist size, and wherein the strap is formed with a rail-like contact electrode for electrical connection between the sensor portion and the display portion.

Unfortunately, the arterial pulse wave detector of this type has a drawback that in order to ensure the positive electrical connection of the contact while permitting the slidable movement of the sensor portion, the pressure on the contact and the selection of a material for the electrode (contact) are limited.

In view of the foregoing, it is an object of the invention to provide a biological information sensing device which is capable of minimizing the effect of the inertia force on the device thereby minimizing the variations of the support for the sensor portion and the displacement thereof, irrespective of the exposure of the device to the accelerative motion during training or other exercises, or of the size of a neck-like portion of a user.

### SUMMARY OF THE INVENTION

In accordance with the invention for achieving the above object, a biological information sensing device applied to a neck-like portion, inclusive of a predetermined measurement area, of a living organism as holding a sensor body thereof in intimate contact with the measurement area for sensing biological information of the living organism, the device comprises a sensor structure comprising the sensor body and a strap-like sensor holder for holding the sensor body; and a strap for fastening the sensor structure to the neck-like portion of the living organism, wherein at least one of an engaging portion of the strap and an engaged portion of the sensor holder extends along a longitudinal direction of the strap for permitting the engaged portion of the sensor holder of the sensor structure to be engagedly secured to the engaging portion of the strap at an optional position with respect to the longitudinal direction of the strap.

The biological information sensing device of the invention is characterized in that the sensor holder of the sensor structure is in the form of a flexible strap serving to hold the sensor body. That is, the biological information sensing device applied to the neck-like portion, inclusive of the predetermined measurement area, of the living organism as holding the sensor body thereof in intimate contact with the measurement area for sensing the biological information of the living organism, the device comprises a flexible sensor structure comprising the sensor body and a flexible strap-like sensor holder for holding the sensor body, and the strap for fastening the sensor structure to the neck-like portion of the living organism, and is characterized in that at least one of the engaging portion of the strap and the engaged portion of the flexible sensor holder extends along the longitudinal direction of the strap for permitting the engaged portion of the flexible sensor holder of the sensor structure to be engagedly secured to the engaging portion of the strap at an optional position with respect to the longitudinal direction of the strap.

According to the biological information sensing device of the invention, the sensor structure comprises the sensor body and the sensor holder in the form of the flexible strap for holding the sensor body and has flexibility. Therefore, the sensor structure can be fastened to the neck-like portion by means of the strap in a fashion that the flexible sensor structure is so placed about the neck-like portion, inclusive of the measurement area, of the living organism as to bring the sensor body into intimate contact with the predetermined measurement area. In addition, at least one of the engaging portion of the strap and the engaged portion of the sensor holder extends along the longitudinal direction in order to permit the engaged portion of the flexible sensor holder of the sensor structure to be engagedly secured to the engaging portion of the strap at an optional position with respect to the longitudinal direction. Accordingly, with the sensor body set at an optimum position for intimate contact with the measurement area, the strap can be so positioned as to be readily and effectively fastened by a fastening hardware irrespective of the size of the neck-like portion. In other words, the sensor body and the fastening hardware of the strap can be placed at optimum positions on the neck-like portion irrespective of the size of the neck-like portion of the user (wearer) of the biological information sensing device. The difference in the size of the neck-like portion may be accommodated by adjusting the position of the sensor holder relative to the strap.

The sensor holder may be formed of a material bendable by hand, such as a urethane resin and soft rubber. However, any other material that has a proper flexibility is also usable. Even a non-flexible material may also be used if it is previously formed in a properly curved shape in conformity with the shape of the arm.

Although a typical example of the neck-like portion is the wrist, the neck-like portion may include other areas of the body such as the neck through which the carotid artery extends.

A typical example of the biological information to be sensed is information on arterial pulses such as frequency of pulse and the like (hence, the biological information sensing device is a so-called arterial pulse wave detector). However, the device of the invention may handle other biological information or signals indicative of blood pressure, serum concentrations of a particular component in blood or indicative of any body fluid other than blood.

According to the biological information sensing device of the invention, the sensor body may be stably held onto or secured to the measurement area so that the biological information (frequency of arterial pulse, variations of blood pressure, level of oxygen in blood or the like) of the user having exercise can be sensed precisely.

According to the biological information sensing device of the invention, the sensor structure typically includes, in addition to the sensor body, an actuating portion for the sensor body and a processing portion for processing the biological information sensed by the sensor body. In this case, the sensor structure may optionally further include a display portion for display of the information processed by the biological-information processing portion. In a case where the display portion is integrated with the sensor structure, the strap may be, for example, provided with a transparent portion or an opening for the view of the display portion, or may be partially decreased in width for permitting the view of the display portion. The sensor structure may include, in place of the display portion, a transmitter portion for non-contact transmission of the information processed by the biological-information processing portion. In this case, a receiver portion for receiving the transmitted information and the display portion may be integrated with the strap for supporting the sensor structure, or otherwise may be formed as a separate article from the strap. In either cases, a sliding contact is not required and hence, the device has a simple structure and is less susceptible to noises, thus achieving an increased operation stability. In addition, the sensor portion and the processing portion for processing the sensed information or signal may be located close to each other and hence, these components are less susceptible to noises, achieving increased operation stabilities.

According to the biological information sensing device of the invention, the engaged portion of the sensor structure and the engaging portion of the strap typically comprise a pair of hook and loop fasteners. Alternatively, either one of the engaged portion of the sensor structure and the engaging portion of the strap may comprise a plurality of recesses distributed along the longitudinal direction whereas the other may comprise a projection engageable with at least one of the recesses. In this case, it is possible to maintain the position of the sensor structure relative to the strap once the sensor structure is positioned on the strap. Accordingly, there is no need for adjusting again the position of the sensor structure in the repeated use of the device by the same user.

In a case where the sensor portion of the biological information sensing device of the invention is a sensor for sensing the blood flowing through the radial artery, the strap is typically designed to be fastened to the wrist by means of a fastening hardware positioned at the cubitus. This provides for an easy and positive fastening of the strap to the wrist so that the sensor may be readily and positively secured to the measurement area as pressed there against at a desirable pressure for the sensor structure.

In a case where the biological information sensing device of the invention comprises an arterial pulse wave detector including an arterial pulse wave sensor based on supersonic wave, the actuating portion for the sensor body of the biological information sensing device typically comprises an oscillating/actuating portion for a supersonic transmitter of the sensor portion, whereas the processing portion for processing the biological information sensed by the sensor body typically comprises an arterial pulse wave receiving portion for extracting an analog arterial pulse signal from the supersonic signal received by the supersonic receiver of the sensor body, and a digital signal processing portion for converting the arterial pulse signal received by the arterial pulse wave receiving portion into a digital signal and processing the resultant digital signal. These information or signal processing portions are formed on individual separate rigid or flexible circuit boards as blocks, which are interconnected into a strap form by means of a flexible cable or the like. In order that the masses are uniformly distributed, at least a part of the information or signal processing portion may be assigned to an additional block. Conversely, at least some of the information or signal processing portions maybe combined into one block. It is noted that a power source like a battery, which is prone to heavy weight, may be positioned at a suitable position for uniform mass distribution. As required, the physical positions of the individual components may optionally be selected. For instance, plural batteries as the power source may be distributed along the strap-like sensor holder.

### BRIEF DESCRIPTION OF THE DRAWINGS

FIGS. 1 are groups of diagrams showing an arterial pulse wave detector according to one preferred embodiment of the invention, FIG. 1A representing a side view explanatory of a state prior to the engagement between a sensor structure and a strap which constitute an arterial pulse wave detector of a first embodiment, FIG. 1B representing a side view explanatory of a state where the mutually engaged sensor structure and strap constituting the arterial pulse wave detector of FIG. 1A are yet to be worn on the wrist, FIG. 1C representing a side view (section) explanatory of a state where the arterial pulse wave detector of FIG. 1A is worn on the wrist, FIG. 1D representing a side view (section) resemblent to FIG. 1C and explanatory of a state where an arterial pulse wave detector according to a second preferred embodiment of the invention is worn on the wrist, FIG. 1E representing a side view (section) resemblent to FIG. 1C and explanatory of a state where an arterial pulse wave detector according to a third preferred embodiment of the invention is worn on the wrist, and FIG. 1F representing a side view, in section, of a wrist on which the arterial pulse wave detector is worn;

FIG. 2 are groups of diagrams more specifically showing the arterial pulse wave detector according to the second embodiment of the invention, FIG. 2A representing a perspective view explanatory of the sensor structure of the arterial pulse wave detector of the second embodiment of the invention shown in FIG. 1D, FIG. 2B representing an enlarged sectional view explanatory of a similar state to that shown in FIG. 1D, FIG. 2C representing a front view explanatory of an area near a display portion of the arterial pulse wave detector shown in FIG. 2B, FIG. 2D representing a similar front view to FIG. 2C for illustrating a modification of FIG. 2C;

FIGS. 3 are groups of block diagrams illustrating the functions of the arterial pulse wave detectors shown in. FIGS. 1 and 2, FIG. 3A representing a functional block diagram showing an example of the arterial pulse wave detector shown in FIG. 1D and the like, and FIG. 3B representing a functional block diagram showing a modification of FIG. 3A;

FIG. 4 are groups of schematic time charts of signals processed by the arterial pulse wave detectors shown in FIGS. 1 to 3, FIG. 3A representing a transmitted supersonic signal, FIG. 3B representing a received supersonic signal modulated in frequency by the Doppler effect, FIG. 3C representing an amplitude modulated signal obtained by differential amplification, and FIG. 3D representing a signal of an extracted amplitude component;

FIG. 5 is a perspective view explanatory of a sensor portion employed by the arterial pulse wave detectors shown in FIGS. 1 to 4; and

FIG. 6 are groups of diagrams showing a modification of engagement means for engaging a sensor holder with the strap, FIG. 6A representing a side view explanatory of a portion including engaging projections, FIG. 6B representing a perspective view explanatory of a portion including an engaged hole.

### DESCRIPTION OF THE PREFERRED EMBODIMENTS

Some of the preferred modes of the invention will be described with reference to the preferred embodiments thereof shown in the accompanying drawings.

### EXAMPLES

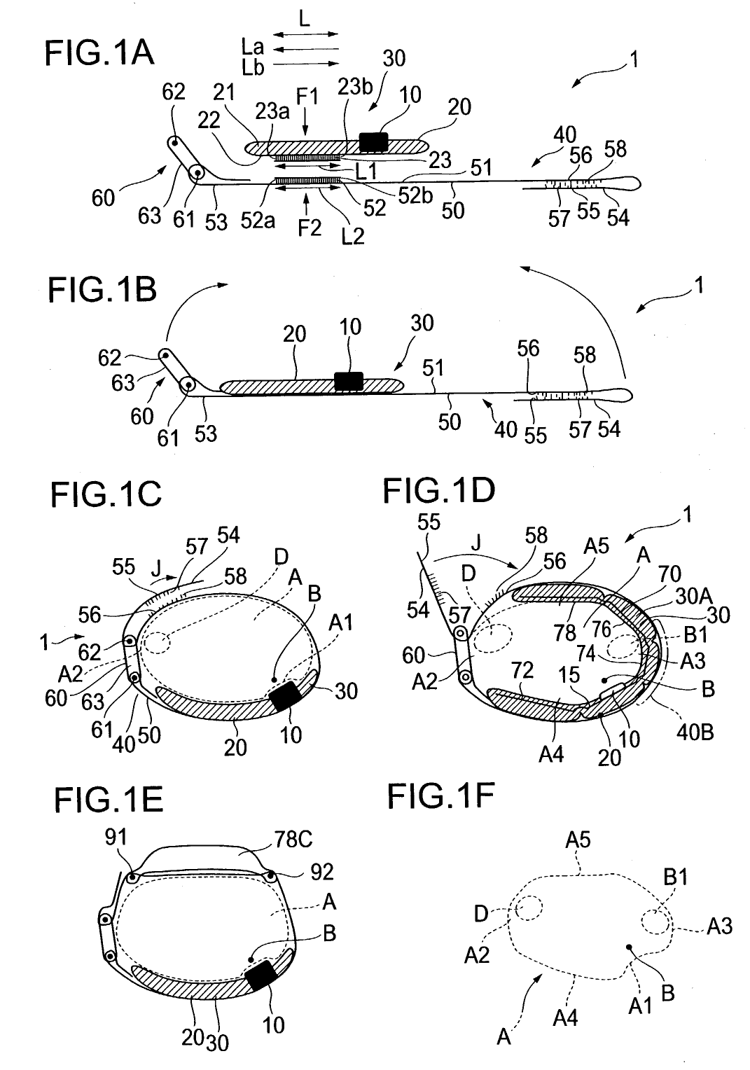

As shown in FIG. 1A, an arterial pulse wave detector 1 as the biological information sensing device of a preferred embodiment of the invention comprises a flexible sensor structure 30 including a sensor body 10 and a sensor holder 20 in the form of a flexible strap serving to hold the sensor body 10; and a strap 40 for fixing the sensor structure 30 to a wrist A. The flexible strap-like sensor holder 20 of the sensor structure 30 is provided with a hook fastener 23 on an outside surface 22 of a flexible strap piece 21, the hook fastener 23 extending a length L1 along a longitudinal direction or an extension direction L of the strap piece. The strap 40 includes a flexible strap body 50, and a fastening hardware structure 60. The strap body 50 is provided with a loop fastener 52 on an inside surface 51 thereof, the loop fastener formed in complementary relation with the hook fastener 23 for engagement therewith and extending a length L2 along the longitudinal direction or extension direction L of the strap body. The pair of hook and loop fasteners 23, 52 in face-to-face relation are pressed against each other along respective directions F1, F2 and secured to each other, thereby securing the sensor structure 30 to the strap 40 including the strap body 50. The hook and loop fasteners 23, 52 extend the respective lengths L1, L2 along the longitudinal direction L. Thus, within a range between a position where an La-wise end 23a of the hook fastener 23 opposes an Lb-wise end 52b of the loop fastener 52 and a position where an Lb-wise end 23b of the hook fastener 23 opposes an La-wise end 52a of the loop fastener 52, the hook fastener 23 is at least partially engageable with the loop fastener 52 even though the hook fastener 23 is shifted relative to the loop fastener 52. Therefore, the hook fastener 23 can be secured to the loop fastener 52. Accordingly, the sensor structure 30 is fixable to the strap 40 with its position adjusted generally in the range of L1+L2 along the L-direction. In the illustrated example, the hook fastener 23 is disposed at a place shifted from a back side of the sensor body 10 along the L-direction. However, the hook fastener 23 may be so located as to overlap the back side of the sensor body 10, so long as a pressure for pressing the sensor body 10 against the wrist portion A can be constantly maintained within a predetermined range.

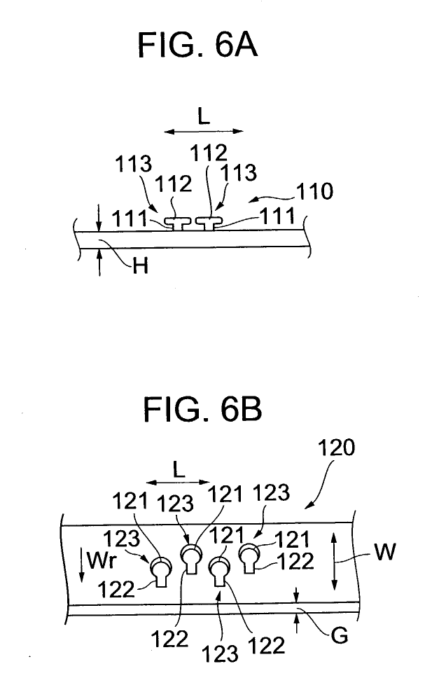

It is noted that the pair of hook and loop fasteners 52, 23 may be replaced by another desirable pair of engagement means so long as the engagement means permit the sensor holder 20 of the sensor structure 30 to be engaged with and fixed to the strap 40 at any practically optional position along the longitudinal direction L of the strap 40. Such a pair of engagement means may be a combination of, as shown in FIGS. 6A and 6B, an engaging portion 110 having a mushroom-like projection 113 including a column-like shaft 111 having a disc-like head of a greater diameter 112 at its distal end, and an engaged portion 120 having a plurality of engaged hole 123 arranged along the longitudinal direction L, the engaged hole 123 including a circular hole 121 for disengageably receiving the greater-diameter head 112 of the mushroom-like projection 113, and a notch 122 formed at place of a circumference of the circular hole 121 for fittingly receiving the column-like shaft 111. In a case where a portion formed with the hole 123 (the strap body 50 of the strap 40, for example) has a thickness G practically smaller than a height H of the shaft 111, the greater-diameter head 112 of the projection 113 of the engaging portion 110 may be inserted into the circular hole 121 of the engaged hole 123 of the engaged portion 120 to bring the column-like shaft 111 into fitting engagement, and then the engaging portion 110 may be moved along a Wr-direction relative to the engaged portion 120 thereby bringing the column-like shaft 111 of the projection 113 of the engaging portion 110 into engagement with the notch 122 of the engaged portion 120. It is noted herein that either one of the pair of components engaging with each other or locking to each other is referred to as “the engaging portion”, while the other is referred to as “the engaged portion”. In this case, a typical practice is to form the engaged portion 120 at the strap 40 and to form the engaging portion 110 at the sensor holder 20 of the sensor structure 30. However, in a case where a slit is formed at a portion of the sensor structure 30 in the direction of its thickness, the engaged portion 120 including the engaged hole 123 may be formed in the sensor structure 30. It is noted that the shape and distribution of the projection 113 constituting the engaging portion 110 as well as the shape and distribution of the hole 123 constituting the engaged portion 120 may be changed as desired.

In the example shown in FIG. 1 and the like, the fastening hardware structure 60 of the strap 40 has a fastening hardware 63 including a pair of pins or shafts 61, 62. One 61 of the shafts of the fastening hardware 63 is engaged with a loop-like end 53 of the strap body 50 whereas the other shaft 62 thereof is engageable with the other end 54 of the strap body 50. A pair of hook and loop fasteners 57 and 58 are also provided on surface portions 55 and 56 of the end 54 of the strap body 50. The hook fastener 57 on the surface portion 55 is pressed against the confronting loop fastener 58 on the surface portion 56 thereby fixing the end 54 of the strap body 50. Cylindrical peripheries of the shafts 61, 62 are typically rotatable about respective axes of the shafts.

When the arterial pulse wave detector 1 is used on the wrist A of the user, the following procedure may be taken to assemble the arterial pulse wave detector 1 as shown in FIG. 1B, for example. The sensor structure 30, shown in FIG. 1A, is fixed to the strap 40 at a desired position with respect to the L-direction according to the size of the user's wrist A (FIG. 1C) by means of the hook and loop fasteners 23, 52. Subsequently, as shown in FIG. 1C, the sensor structure 30 is positioned in a manner that the sensor body 10 is pressed against a surface area A1 of the wrist A where the radial artery B is close to the wrist surface. Then, after threading the end 54 of the strap body 50 through the fastening hardware 63, the detector 1 is so positioned as to bring the shaft 62 into contact with a bump area A2 corresponding to the cubitus D. Subsequently, the strap 40 is tightened by pulling the end 54 of the strap body 50, and the hook and loop fasteners 57, 58 at the end 54 are pressed against each other thereby fixing the strap body 50. The L-direction position of the sensor structure 30 relative to the strap 40 may be selectively set such that with the sensor body 10 positioned to confront the surface area A1 of the wrist near the radial artery B, the rotary shaft 62 may correspond to the bump area A2 at the cubitus D. This provides for an easy and positive fastening of the strap 40.

Although the sensor structure 30 of the example shown in FIGS. 1A to 1C has a length of about less than a half of an outside circumference of the wrist portion A, the sensor structure 30 may have a greater length than this. As shown in FIG. 1D, for instance, the sensor structure may have a length of about ¾ or more of the outside circumference of the wrist portion A. In the case of a sensor structure 30A of the greater length as shown in FIG. 1D, the elongate flexible sensor structure 30A is disposed in a manner to overlap the strap body 50 except for a region where the fastening hardware structure 60 of the strap 40 exists.

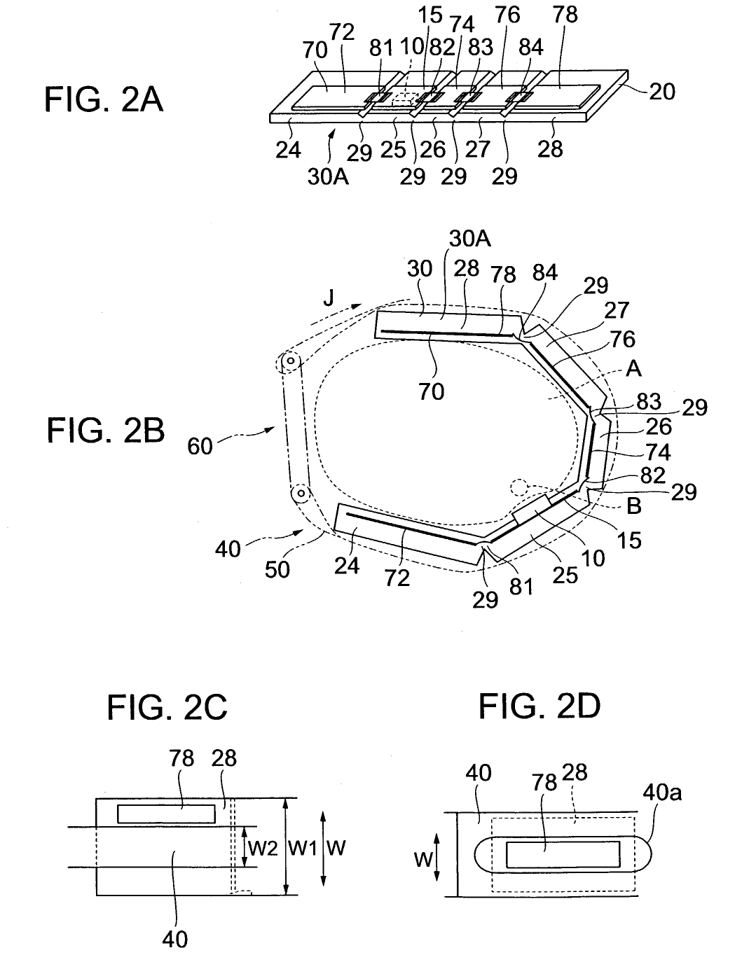

Specifically, of the wrist A substantially of an elliptic or oval section, an area A3 having a relatively great curvature near the radius B1 as well as areas A4, A5 on both sides thereof having smaller curvatures are used as portions along which the sensor structure 30 extends, so that the area A2 of a great curvature near the bubitus D may be utilized for fastening the strap to place by means of the fastening hardware structure 60. This permits the strap 40 to be effectively fastened to place. As indicated by a phantom line 40B in FIG. 1D, for example, a part of the strap 40 may comprise an elastic strap piece 40b such as of a rubber material in order that the strap 40 may present a generally constant fastening force. It goes without saying that such a strap including the elastic strap piece may also be used for securing the sensor structure 30 shown in FIGS. 1A to 1C.

Where the sensor structure 30 is the sensor structure 30A having the length to surround the most part of the wrist A as shown in FIG. 1D, a circuit portion 70 of the sensor structure 30A comprises, as shown in FIG. 2A for example, the following components arranged in blocks in addition to an arterial pulse wave sensor portion 15 comprising the sensor body 10 including a supersonic transmitter 11 and a supersonic receiver 12, the components including an oscillating/actuating portion 72 for the supersonic transmitter 11 of the sensor body 10; an arterial pulse wave receiving portion 74 for extracting an analog arterial pulse signal from a supersonic signal received by the supersonic receiver 12 of the sensor body 10; a digital signal processing portion 76 for converting the arterial pulse signal extracted by the arterial pulse wave receiving portion 74 into a digital signal and processing the resultant digital signal; and a display portion 78 for displaying the processing results given by the digital signal processing portion 76 (incidentally, the sensor structure 30 shown in FIGS. 1A to 1C typically includes the same circuit portion 70, as well).

The circuit components 72, 25, 74, 76 and 78 constituting the circuit portion 70 of the sensor structure 30 each comprise a circuit board and a circuit device incorporated in the circuit board. The circuit components are each connected with the respective adjoining circuit component 15, 74, 76 or 78 via a respective flexible cable 81, 82, 83 or 84. It is noted here that each of the circuit boards may be a printed wiring board such as formed of a resin or ceramic, or a circuit board per se forming an integrated circuit board. In the examples shown in FIGS. 2A, 2B and 1D, the circuit boards are typically rigid, but the circuit boards themselves may have flexibility.

The sensor holder 20 of the sensor structure 30 is formed of a flexible strap material such as a urethane resin, and includes, for example, strap forming bases 24, 25, 26, 27 and 28 individually serving to support their respective circuit components 72, 15, 74, 76 and 78, and interconnection portions 29 for interconnecting the forming bases 24 to 28. FIGS. 2A and 2B illustrate the example where the sensor holder 20 is formed of a material having a greater rigidity than that of the sensor holder shown in FIG. 1D. The degree of flexibility of the sensor holder 20 may be selected according to requirements with the proviso that the sensor holder 20 is soft or flexible enough to permit the sensor body 10 to be positioned/fixed. The strap forming bases 24, 25, 26, 27 and 28 of the sensor holder 20 have the corresponding circuit components 72, 15, 74, 76 and 78 laid thereon or embedded therein. In this connection, the strap forming bases 24, 25, 26, 27 and 28 are previously formed with recesses or openings on either one surface or both surfaces thereof for receiving therein the corresponding circuit components 72, 15, 74, 76 and 78 such that the circuit components 72, 15, 74, 76 and 78 maybe disposed/fixed therein. For instance, the sensor body 10 may be so disposed on the associated portion 25 of the sensor holder 20 as to project from an inside surface of the sensor holder 20, whereas the circuit component 78 including the display portion may be so disposed on the associated portion 28 as to be visually recognized from an outside surface of the sensor holder 20. Such a structure may be constructed, for example, by placing the circuit components 72, 15, 74, 76, and 78 in the corresponding recesses and then interconnecting the adjoining circuit components by means of the respective flexible cables 81, 82, 83 and 84. If desired, of course, at least some or all of the circuit components 72, 15, 74, 76 and 78 may be formed integrally with the corresponding strap forming bases 24, 25, 26, 27 and 28 at the forming of the strap forming bases 24, 25, 26, 27 and 28 so that the circuit components are embedded in the corresponding strap forming bases 24, 25, 26, 27 and 28 of the sensor holder 20.

As shown in FIG. 5 for example, the sensor portion 15 comprises a common substrate 14 incorporating therein the sensor body 10 including the supersonic transmitter 11 and the supersonic receiver 12 individually including a piezoelectric device.

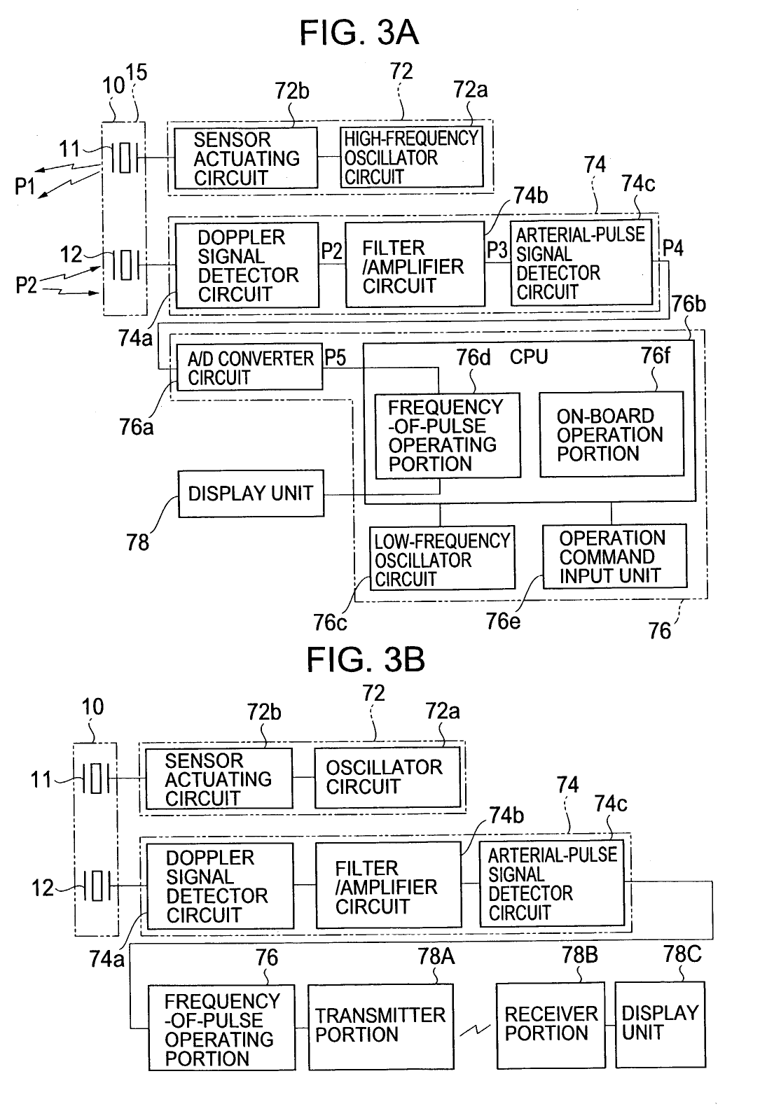

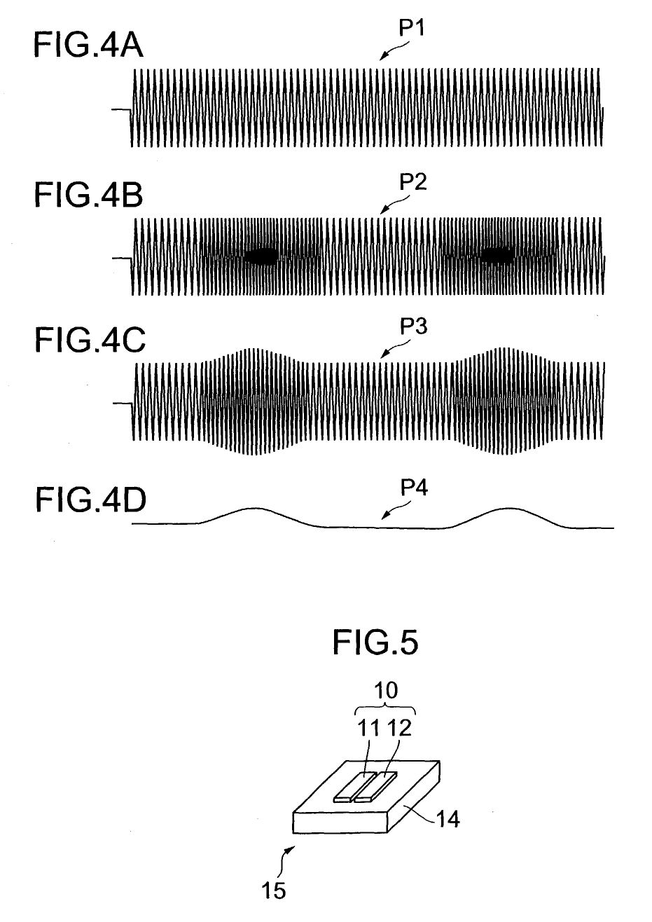

In the arterial pulse wave detector 1, as shown in FIG. 3A, the supersonic transmitter 11 of the sensor body 10 is actuated to transmit a supersonic signal P1 under the control of the oscillating/actuating circuit portion 72 including a high-frequency oscillator circuit 72a and a sensor actuating circuit 72b while the signal P1 is reflected as impinging upon blood components, such as blood cells or the like, in blood flowing through the radial artery B. The supersonic signal emitted from the supersonic transmitter 11 is typically the signal P1 practically having a constant frequency and amplitude, as shown in FIG. 4A, for example.

A supersonic signal P2 reflected by the blood components in blood as a pulsing stream through the radial artery B and received by the supersonic receiver 12 is modulated in frequency due to the Doppler effect associated with the pulse of the blood components as the reflector of the transmitted supersonic signal P1. Hence, the signal P2 assumes a form as shown in FIG. 4B, for example.

The arterial pulse wave receiving portion 74 for extracting an analog arterial pulse signal P4 from the supersonic signal P2 received by the supersonic receiver 12 of the sensor body 10 includes, for example, a doppler signal detector circuit 74a, a filter/amplifier circuit 74 band an arterial pulse signal detector circuit 74c, as shown in FIG. 3A. An output from the doppler signal detector circuit 74a is, for example, an electrical signal of a similar wave form P2 to that of the received supersonic signal P2. The filter/amplifier circuit 74b amplifies an amount of variation of the doppler signal P2˜sin{(ω+Δω)t} using the original transmission signal P1˜sin(cot) as a reference or a reference signal, so as to extract a differential amplification signal P3˜{sin(Δω/2)t}·sin{(Δω/2)}t as shown in FIG. 4C. It is noted here that ω denotes an angular frequency of the supersonic signal P1, and that Δω=Δω(t) denotes a modulated angular frequency dependent upon time t due to the Doppler effect. In the arterial pulse wave receiving portion 74, the arterial pulse signal detector circuit 74c extracts, as the arterial pulse signal P4, an amplitude modulated component from the differential amplification signal P3. In the case of a square law detection, the arterial pulse component P4 can be extracted as sin{(Δω)t}.

Although FIG. 4 show the arterial pulse wave P4 quite in a simple wave form, the arterial pulse wave P4 actually presents much more complicated time-dependent wave form than that of FIG. 4D. Particularly in a state where the cardiopulmonary circulatory system is overtaxed during or after exercise, the arterial pulse wave assumes a much more complicated and irregular wave form containing a wide range of high frequency components.

In the case of an arterial pulse wave detector 1 shown in FIG. 3A, the digital signal processing portion 76 includes an analog/digital (A/D) converter circuit 76a for converting the analog signal P4 indicative of the arterial pulse wave into a digital signal P5 indicative of the arterial pulse wave; a central processing unit (CPU) 76b for receiving the digital arterial pulse signal P5; and a low-frequency oscillator circuit 76c for supplying the CPU 76b with a reference signal for processing. In this case, the CPU 76b includes a memory for storing a frequency-of-pulse operation program and a microprocessor for executing the program, thus forming a frequency-of-pulse operating portion 76d for operating the frequency of pulse based on the digital arterial pulse signal P5 with reference to the low-frequency signal from the low-frequency oscillator circuit 76c. Typically, the CPU forms a digital signal processor (DSP) wherein a part of the frequency-of-pulse operation program including a fast Fourier transformation (FFT) process is incorporated in a digital signal processor circuit. It is noted that the CPU 76b further includes a device operation portion 76f for receiving an operation command from an operation command input portion 76e such as a push-button switch.

According to FIG. 3A, the display portion 78 comprises a display unit for displaying the operation result or frequency of pulse Q determined by the frequency-of-pulse operating portion 76d of the CPU 76b.

In a case where the sensor structure 30 or 30A includes the display portion 78, the strap forming base 28 including the display portion 78 may be provided with the display portion 78 at place near one end thereof with respect to a width-wise direction W thereof as shown in FIG. 2C or at a central portion thereof with respect to the width-wise direction W thereof as shown in FIG. 2D. In the former case, as shown in FIG. 2C for example, a region of the strap 40, that is overlapped by the strap forming base 28, is formed in an adequately smaller width W2 than a width W1 of the strap forming base 28 in order to permit the view of the display on the display portion 78. Alternatively, the strap 40 may be formed in a width equal to or greater than that of the strap forming base 28 and a side edge portion of the strap forming base 28, that is overlapped by the display portion 78, maybe formed of a transparent material. In the latter case, the strap 40 includes an opening 40a at its region overlapped by the display portion 78 of the strap forming base 28 in order to permit the view of the display on the display portion 78 disposed centrally with respect to the width-wise direction W of the strap, as shown in FIG. 2D. Of course, the opening 40a may be formed of a transparent material.

According to the foregoing description, the A/D converter circuit 76a, the operation command input portion 76e and the like belong to the digital signal processing portion 76. However, the A/D converter circuit 76a may belong to, for example, an output circuit portion of the arterial pulse wave receiving portion 74 for processing the analog signal. Further, the operation command input portion 76e like a push-button switch may be integrally formed with the display unit 78 as an article. Similarly, the other components may be freely combined into blocks, as desired, so long as such combinations contribute to the mass distribution as a whole.

Needless to say, the CPU 76b may perform other operations during spare-time when the frequency of pulse Q is not operated or at an interval between the operations of the frequency of pulse Q. One example of the other operations include a time counting operation as a clock. Specifically, the CPU 76b is, for example, capable of performing the time counting operation as a timer and hence, the display portion 78 is also capable of functioning as a display of a digital clock.

The arterial pulse wave detector 1 of the above construction may be worn on the wrist A as follows. The arterial pulse wave detector 1 is placed around the wrist A in a manner to bring the sensor body 10 of the sensor portion 15 into abutment against the wrist surface area A1 near the radial artery B of the wrist A. With the fastening hardware 63 of the fastening hardware structure 60 positioned at the wrist bump area A2 near the cubitus D, the strap piece 54 is threaded through the fastening hardware 63 and pulled along a J-direction. Then, the strap piece 54 is fixed by means of the hook and loop fasteners 57, 58.

When the arterial pulse wave detector 1 is wrapped around the wrist in this manner, the sensor structure 30 of the arterial pulse wave detector 1 has substantially uniform mass distribution along the longitudinal direction thereof. Therefore, even when the wrist A is subjected to the accelerative motion due to exercise or the like, such a great inertia force as to bring the arterial pulse wave detector 1 into mono-directional rotation about the wrist A will not actually occur. Accordingly, the sensor body 10 of the sensor portion 15 of the arterial pulse wave detector 1 is maintained in intimate contact with the measurement area A1, thus achieving the precise measurement of the arterial pulses. Since the sensor body 10 and the display portion 78, as a part of the circuit portion 70, are unified as one piece, mediation of a sliding contact and the like is dispensed with. Thus, there is no fear of noises during the signal processings.

Another approach may replace the direct display of the frequency of pulse Q on the display portion 78 shown in FIG. 3A. As shown in FIG. 3B, a transmitter portion 78A including an antenna or coil is adapted to transmit the data on the frequency of pulse Q, obtained by the frequency-of-pulse operating portion 76d, in the form of an electromagnetic signal R such as of an electromagnetic wave or variable magnetic field, whereas a separate receiver portion 78B receives the electromagnetic signal R, from which the frequency of pulse Q is extracted to be displayed on a display unit 78C.

In this case, the arterial pulse wave detector worn on the wrist of one arm takes the form of a headless strap while, for example, the frequency of pulse Q may be displayed on the display unit 78C with a clock function which is worn on the other arm. This permits the whole body of the display unit 78C prone to heavy weight to be mechanically separated from an arterial pulse wave detector 1B, contributing to the weight reduction and mass distribution of the arterial pulse wave detector 1B. As a result, the possibility of variations of the pressure for pressing the sensor body 10 of the arterial pulse wave detector 1B against the wrist A or displacement of the sensor body 10 of the detector 1B relative to the area Al of the wrist A can be reduced.

In this case, as well, the display unit 78C having the clock and other functions may be adapted to be worn on the wrist A by means of the strap 40 having a pair of pins 91, 92 pivotally movable about their axes, as shown in FIG. 1E for example.

The arterial pulse wave detectors shown in FIGS. 1A to 1C and 1E are practically constructed the same way as the arterial pulse wave detectors shown in FIGS. 1D, 2A and 2B, except that the sensor structure has a smaller length than that of the arterial pulse wave detectors of FIGS. 1D, 2A and 2B, and that the circuit boards constituting the sensor structure are generally unified into one piece by a more flexible sensor holder 20. If desired, however, a greater part of the circuit portion involved in the signal processings and the like may be separated from the sensor structure 30 so that the sensor structure 30 may have a relatively smaller size.

What is claimed is:

1. A biological information sensing device comprising:
a sensor structure comprising a sensor body for sensing biological information of a living organism in intimate contact with a predetermined measurement area and a strap-like sensor holder for holding the sensor body; and
a strap for fastening the sensor structure to the neck-like portion of the living organism;
wherein at least one of an engaging portion of the strap and an engaged portion of the sensor holder extends along a longitudinal direction of the strap for permitting the engaged portion of the sensor holder of the sensor structure to be engagedly secured to the engaging portion of the strap at an optional position with respect to the longitudinal direction of the strap.

2. A biological information sensing device as claimed in claim 1, wherein the sensor holder of the sensor structure is in the form of a flexible strap serving to hold the sensor body.

3. A biological information sensing device as claimed in claim 1, wherein the sensor structure further includes, in addition to the sensor body, an actuating portion for the sensor body and a processing portion for processing the biological information sensed by the sensor body.

4. A biological information sensing device as claimed in claim 3, wherein the sensor structure includes a display portion for display of the information processed by the biological-information processing portion.

5. A biological information sensing device as claimed in claim 3, wherein the sensor structure includes a transmitter portion for non-contact transmission of the information processed by the biological-information processing portion.

6. A biological information sensing device as claimed in claim 1, wherein the engaged portion of the sensor structure and the engaging portion of the strap comprise a pair of hook and loop fasteners.

7. A biological information sensing device as claimed in claim 1, wherein either one of the engaged portion of the sensor structure and the engaging portion of the strap comprises a plurality of recesses distributed along the longitudinal direction whereas the other comprises a projection engageable with at least one of the recesses.

8. A biological information sensing device as claimed in claim 1, wherein the sensor portion is a sensor for sensing information on blood flowing through the radial artery, and wherein the strap is designed to be fastened to the wrist via a fastening hardware positioned at the cubitus.
